(19) United States
(12) Patent Application Publication
(10) Pub. No.: US 2014/0051631 A1
(43) Pub. Date: Feb. 20, 2014

(54) PHARMACEUTICAL COMPOSITION COMPRISING TPO OR AN AGONIST OF THE TPO RECEPTOR

(71) Applicants: Francoise Porteu, Paris (FR); Patrycja Pawlikowska, Paris (FR); Berengere de Laval, Paris (FR)

(72) Inventors: Francoise Porteu, Paris (FR); Patrycja Pawlikowska, Paris (FR); Berengere de Laval, Paris (FR)

(21) Appl. No.: 13/587,973

(22) Filed: Aug. 17, 2012

(51) Int. Cl. A61K 38/22; G01N 33/574; C12Q 1/68; A61P 35/00

(52) U.S. Cl. 514/7.8; 435/7.23; 435/6.12

(57) ABSTRACT

The present invention relates to a new use of TPO or a TPO/mpl receptor agonist as an adjuvant of a treatment of a neoplastic disease by irradiation or chemotherapeutic agents in order to prevent genomic instability of hematopoietic stem cells incurred through radio- or chemotherapy and hence to prevent the occurrence of secondary cancers, namely acute myelogenous leukemia and myelodisplasia.

### FIELD OF THE INVENTION

The instant invention relates to the general field of cancer therapy and prognosis of cancer.

### BACKGROUND OF THE INVENTION

Double-strand breaks (DSBs) in genomic DNA are generated endogenously during normal cellular processes such as replication or can arise through exogenous treatments with a variety of genotoxic agents such as ionizing radiation and topoisomerase inhibitors. In mammalian cells, DSBs are removed by two main repair pathways: homologous recombination (HR) and non-homologous end joining (NHEJ). Both mechanisms complement each other, are strictly regulated and highly conserved in evolution. HR is a process in which a homologous DNA strand is used as a template for the repair of DSB and therefore its use is restricted to late S and G2 phase of the cell cycle. NHEJ requires no template and mediates end-ligation after the processing of the broken DNA ends. It is thought to operate throughout the entire cell cycle but is used preferentially for the removal of DSBs arising in GO and GI. This pathway is regarded as the predominant mechanism for DSB repair in vertebrates.

Central to NHEJ are the DNA-PK and DNA ligase IV/XRCC4/XLF complexes. In contrast to HR, NHEJ does not ensure the restoration of the original sequence and therefore NHEJ is considered intrinsically error prone. However, it is admitted that the extremely rapid processing favors synapsing of correct ends and suppresses the formation of exchange type aberrations. Studies of mice or cells null for key NHEJ canonical components have shown that this pathway plays a crucial role as a caretaker of the mammalian genome, its deficiency leading to major DSB-induced genome rearrangements, including translocations and deletions in mammalian cells.

Bone marrow (BM) injury is one of the major limiting factors for cancer therapies of anticancer DNA-damaging agents, such as ionizing radiations (IR) and chemotherapeutic drugs. Apart from the acute myelosupression resulting from the death of rapidly proliferating hematopoietic progenitor cells (HPCs), a large portion of patients also develop long-term BM injury due to loss of hematopoietic stem cell (HSC) numbers or function. In addition, IR induced risk of cancer development is very high for the hematopoietic tissue. Indeed, secondary acute myeloid leukemia or myelodysplasia are the major adverse complications of chemotherapy or radiotherapy used to treat highly curable malignancies such as breast cancers, Hodgkin's disease or childhood acute lymphoid leukemia. Defective DNA repair has been associated with a spectrum of blood disorders and the occurrence of chromosomal translocations is the hallmark of human hematological malignancies. Since they maintain hematopoietic homeostasis throughout life-span trough self-renewal and differentiation, HSCs have an increased risk of accumulating genomic anomalies that are likely to compromise their genomic integrity and potentially give rise to cancer. This makes HSCs the major targets for radiation induced carcinogenesis. Maintenance of genomic integrity is also crucial for the preservation of the self-renewal capacity of HSCs. Therefore, approaches for improving the safety of anti-cancer DNA agents require a careful analysis of HSC response to DNA damage insults. Previous studies have shown that HSCs are more resistant than their downstream myeloid progeny in response to ionizing radiation (IR), but only limited data are available about the mechanisms involved in DNA repair in HSCs. Recently, it has been shown that DSB repair through NHEJ appears necessary to stem cell maintenance, as mice deficient for DNA Ligase IV show a progressive loss of HSC function upon aging. However, the mandatory use of NHEJ-mediated IR-induced DNA damage in quiescent HSCs has also been shown to promote error-prone DNA repair and mutagenesis. HSC are also regulated though interaction with the niche environment and by a variety of growth factors which regulate the balance between quiescence, proliferation and differentiation. How these signals intervene on the intrinsic DNA repair process of HSC and progenitors is currently unknown.

Thrombopoietin (TPO) and its receptor TPO-R (or Mpl) is the master regulator of megakaryopoiesis and platelet production. Mpl is expressed on long-term HSCs and Mpl signaling also plays a key role in these cells. Indeed, TPO has been shown to support their quiescence and to protect the HSC pool from premature exhaustion, as demonstrated by the increased cycling and age progressive loss of HSCs in TPO−/− and Mpl−/− mice. Inactivating mutations of the Mpl receptor in humans cause thrombocytopenia and bone marrow failure in early childhood, frequently leading to early death of patients, a syndrome called congenital amegakaryocytic thrombocytopenia. On the contrary, mutations resulting in constitutive Mpl activation are involved in myeloproliferative disorders.

### SUMMARY OF THE INVENTION

The present invention relates to a new use of TPO or a TPO/mpl receptor agonist as an adjuvant of a treatment of a neoplastic disease by irradiation or chemotherapeutic agents in order to prevent genomic instability of hematopoietic stem cells incurred through radio- or chemotherapy and hence to prevent the occurrence of secondary cancers, namely acute myelogenous leukemia and myelodisplasia.

According to a first aspect, the present invention provides a pharmaceutical composition comprising TPO or an active fragment thereof or an agonist of the TPO receptor (TPO-R) for preventing genomic instability of hematopoietic stem cells incurred through radio- or chemotherapy in the course of the treatment of a neoplastic disease with the exception of a myeloid hemopathy, wherein said pharmaceutical composition is administered prior to the radio- or chemotherapy treatment.

The pharmaceutical composition of the invention is preferably administered to a subject in need of a radiotherapy or chemotherapy treatment of a neoplastic disease or disease in a single dose prior to the administration of the radio- or chemotherapy treatment.

According to one aspect the pharmaceutical composition comprises TPO or an active fragment thereof.

According to another aspect, the pharmaceutical composition comprises an agonist of the TPO receptor.

Typical examples of agonists of the TPO-R are romiplostim and eltrombopag.

According to a second aspect, the invention provides a method for the prognosis of an acute myelogenous leukemia or myelodisplastic syndrome secondary to a radio- or chemotherapy treatment of a neoplastic disease, comprising measuring the level of expression of the TPO/mpl receptor.

According to a third aspect, the invention relates to a method for treating a neoplastic disease other than a myeloid hemopathy by irradiation and/or administration of (a) chemotherapeutic agent(s), comprising administering to a subject prior to said irradiation and/or administration of said chemotherapeutic agent(s) a composition comprising an effective amount of TPO or an active fragment thereof or an agonist of the TPO receptor.

### DETAILED DESCRIPTION OF THE INVENTION

The inventors of the instant invention have demonstrated that, in addition to its known functions, TPO has the unique capacity among cytokines acting on HSCs, to protect HSPCs from IR and the chemotherapeutic drugs doxorubicin and etoposide (VP16) induced DNA damage through the increase of DNA-PK activity and NHEJ repair efficiency. A single injection of TPO to mice immediately before total body irradiation (TBI) protects HSPCs from acquiring genomic rearrangements and prevents long-term loss of HSPC reserves and function. Mpl−/− mice show increased radio sensitivity due to defective repair function. Mpl expression is haplosufficient for this function, suggesting that it may also act as a tumorsuppressor in response to radio- or chemotherapy.

Accordingly, the present invention provides a pharmaceutical composition comprising TPO or an active fragment thereof or an agonist of the TPO receptor (TPO-R) for preventing genomic instability of hematopoietic stem cells incurred through radio- or chemotherapy in the course of the treatment of a neoplastic disease with the exception of a myeloid hemopathy, wherein said pharmaceutical composition is administered prior to the radio- or chemotherapy treatment, preferably in a single dose prior said treatment.

The invention further provides a method for the prognosis of an acute myelogenous leukemia or inyelodisplastic syndrome secondary to a radio- or chemotherapy treatment of a neoplastic disease, comprising measuring the level of expression of the TPO/mpl receptor.

The invention also relates to a method for treating a neoplastic disease other than a myeloid hemopathy by irradiation and/or administration of (a) chemotherapeutic agent(s), comprising administering to a subject prior to said irradiation and/or administration of said chemotherapeutic agent(s) a composition comprising an effective amount of TPO or an active fragment thereof or an agonist of the TPO receptor.

### Definitions

Throughout the specification, several terms are employed and are defined in the following paragraphs.

Thrombopoietin is a glycoprotein having at least two forms with apparent molecular masses of 251 kDa and 31 kDa, with a common N-terminal amino acid sequence. Baatout, Haemostasis, 27: 1-8 (1997); Kaushansky, New Engl. J. Med., 339: 746-754.

The gene encoding TPO has been cloned and characterized (Kuter et al., Proc. Natl. Acad. ScL USA. 91: 11104-11108 (1994): Barley et al., Cell, 72:1117-1124 (1994): Kaushansky et al., Nature, 369:568-571 (1994); Wendling et al., Nature, 369: 571-574 (1994); and Sauvage et al., Nature, 369: 533-538 (1994).

Thrombopoietin appears to have two distinct regions separated by a potential Arg-Arg cleavage site. The amino-terminal region is highly conserved in man and mouse, and has some homology with erythropoietin and interferon-β and interferon-β. The carboxy-terminal region shows wide species divergence. The DNA sequences and encoded peptide sequences for human TPO receptor (TPO-R; also known as c-mpl) have been described. (Vigon et al., Proc. Natl. Acad. Sci. USA, 89: 5640-5644 (1992).

TPO-R is a member of the hematopoietin growth factor receptor family, a family characterized by a common structural design of the extracellular domain, including for conserved C residues in the N-terminal portion and a WSXWS motif close to the transmembrane region. (Bazan, Proc. Natl. Acad. Sci. USA, 87: 6934-6938 (1990)). Evidence that this receptor plays a functional role in hematopoiesis includes observations that its expression is restricted to spleen, bone marrow, or fetal liver in mice (Souyri et al., Cell, 63: 1137-1147 (1990)) and to megakaryocytes, platelets, and CD34+ cells in humans (Methia et al., Blood, 82: 1395-1401 (1993)). Further evidence for TPO-R as a key regulator of megakaryopoiesis is the fact that exposure of CD34+ cells to synthetic oligonucleotides antisense to TPO-R RNA significantly inhibits the appearance of megakaryocyte colonies without affecting erythroid or myeloid colony formation. Some workers postulate that the receptor functions as a homodimer, similar to the situation with the receptors for G-CSF and erythropoietin (Alexander et al., EMBO J., 14: 5569-5578 (1995)). The slow recovery of platelet levels in patients suffering from thrombocytopenia is a serious problem, and has lent urgency to the search for a blood growth factor agonist able to accelerate platelet regeneration (Kuter, Seminars in Hematology. 37: Supp 4: 41-49 (2000).

The term [symbol] TPO active fragment” in the sense of the instant invention means a fragment of TPO capable of retaining the protective activity of TPO against DNA double strand breaks or otherwise preventing genetic instability.

The term “TPO receptor agonist” refers to a compound with a biological activity that is known to result, either directly or indirectly from the presence of TPO. Exemplary TPO activities include, but are not limited to, proliferation and or differentiation of progenitor cells to produce platelets; hematopoiesis; growth and/or development of glial cells; repair of nerve cells; and alleviation of thrombocytopenia.

Herein, “hematopoietic stem cells” refers to cells that can differentiate into any type of lymphoid cells or myeloid cells. There is no particular limitation on the hematopoietic stem cells of the present invention, as long as they have the characteristics described above; however, the cells include CD34-positive hematopoietic cells. The CD34-positive hematopoietic cells are a heterogeneous cell population containing CD34-positive hematopoietic stem cells and CD34-positive hematopoietic precursor cells. The CD34-positive hematopoietic cells include, for example, multipotent stem cells, lymphoid stem cells, CFU-GEMM, CFU-GM, BFU-E, and CFU-MEG. Herein, “CD34-positive hematopoietic precursor cells” refers to CD34-expressing cells that are in the process of differentiation into lymphoid cells (B cells, T cells, and such) or myeloid cells (neutrophils, monocytes, erythrocytes, megakaryocytes, and such), but have yet been directed to differentiate into each lineage, or are at a stage where the direction of cell differentiation cannot be identified morphologically. Whether or not cells express CD34 can be assessed by methods known to those skilled in the art, for example, the method described in the Journal of Hematotherapy 5, 213-226, 1996 (Robert Sutherland et al. The ISHAGE guidelines for CD34 cell determination by Flow Cytometry).

According to a first aspect, the invention relates to a pharmaceutical composition comprising TPO or a fragment thereof or an agonist of the TPO receptor (TPO-R) for preventing genomic instability of hematopoietic stem cells incurred through radio- or chemotherapy in the course the treatment of a neoplastic disease, with the exception of a myeloid hemopathy, wherein said pharmaceutical composition is administered in a therapeutically effective amount prior to the radio- or chemotherapy treatment.

As used herein, the term “effective amount” or “therapeutically effective amount” means that amount of a drug or pharmaceutical agent that will elicit the biological or medical response of a tissue, system or human that is being sought, typically by a clinician.

It is self-evident that the composition is administered as a “therapeutically effective amount” (or simply “effective amount”) which is the amount of the composition that will elicit the biological response, namely the genomic instability of hematopoietic stem cells.

The genomic instability of samples can be assessed for the purposes of establishing a control on the traditional basis of nuclear DNA content using routine methods (e.g., image cytometry) and tissues classified as genomically stable or genomically unstable according to known indices (e.g., Kronenwett et al., Cancer Res, 64(3), 904-9 (2004); Kronenwett et al., Cancer Epidemiol Biomarkers Prey, 15(9), 1630-5 (2006) or as described hereunder.

Genomic instability of HSPCs can be assessed by measuring the presence of gH2Ax foci ,which gH2Ax represents a surrogate for the presence of DNA double stand breaks. The measurement can be easily carried out on small cell samples, either by flow cytometry techniques or by immunofluorescnce on slides of cytospun cells.

The term “prior” applied to the administration of the composition of the invention means that the composition is administered between 24 hour before the irradiation procedure or the chemotherapeutic agent or combination of chemotherapeutic agent(s) and immediately before.

It is in general preferred to administer the composition of the invention not later than 2 hours and most preferably 60 minutes before the radio- or chemotherapy and not later than and 2 minutes and preferably 30 minutes before the radio-or chemotherapy.

The pharmaceutical composition of the invention is effective when administered as a single dose prior to the administration of the radio- or chemotherapy treatment. Administration of a single dose is hence preferred. However, if appropriate, the composition may be administered in several doses.

When there are multiple cycles of radio- or chemotherapy, it is preferable to administer the TPO or active fragment thereof or TPO agonist before each cycle as described above.

The composition of the invention may be administered prior to the treatment of any cancer or type of cancer or neoplastic disease or condition for which irradiation and/or chemotherapeutic treatment are required with the exception of a myeloid hemopathy, where the increase of TPO or the activity of TPO must be avoided.

By myeloid hemopathy is intended acute myeloid leukemia, myelodisplastic syndrome, and myeloproliferative disorders.

Preferred TPO-R agonists are romiplostin and eltrombopag.

The composition of the invention is effective for preventing acute myelogenous leukemia or myelodisplasia secondary to a treatment by irradiation or chemotherapy of a neoplastic disease.

Radiotherapy is typically any kind of radiation-based treatment used for solid cancers such as prostate cancers, breast cancers, or blood cancer such as Hodgkin's and non-Hodgkin's lymphoma.

The chemotherapeutic agent used for the treatment of the neoplastic diseases may be any type of agent well known in the art used for the treatment of cancer alone or in combination with other agents.

The term “neoplastic disease” or “neoplastic disorder” according to the present invention refers to a proliferative disorder or disease caused or characterized by the proliferation of cells, which have lost susceptibility to normal growth control. The term “cancer” according to the present invention includes benign and malign tumors and any other proliferative disorders for example the formation of metastasis. Cancers of the same tissue type in general originate from the same tissue, and are for example divided into different subtypes based on their biological characteristics. Four general categories of cancers are carcinoma, sarcoma, leukemia, and lymphoma. Over 200 different types of cancers are known, and every organ or tissue of the body can be affected.

Specific examples of cancers that do not limit the definition of cancer includes solid tumors, blood born tumors such as leukemia [a verifier], acute or chronic lymphoblastic leukemia, breast cancer, chordoma, craniopharyngioma, endometrial cancer, ependymoma, Ewing's tumor, gastric cancer, germinoma, glioma, glioblastoma, hemangioblastoma, hemangioperycatioma, Hodgkins lymphoma, medulloblastoma, leukaemia, mesothelioma, neuroblastoma, non-Hodgkins lymphoma, pinealoma, retinoblastoma, sarcoma (including angiosarcoma, osteosarcoma and chondrosarcoma), bladder carcinoma, brain tumor, breast carcinoma, bronchogenic carcinoma, carcinoma of the kidney, cervical carcinoma, choriocarcinoma, cystadenocarcinome, embryonal carcinoma, epithelial carcinoma, esophageal carcinoma, cervical carcinoma, colon carcinoma, colorectal carcinoma, endometrial carcinoma, gallbladder carcinoma, gastric carcinoma, head and neck carcinoma, liver carcinoma, lung carcinoma, medullary carcinoma, non-small cell bronchogenic/lung carcinoma, lung cancer, ovarian carcinoma, pancreas carcinoma, papillary carcinoma, papillary adenocarcinoma, prostate carcinoma, small intestine carcinoma, rectal carcinoma, renal cell carcinoma, skin carcinoma, squamous cell carcinoma, sebaceous gland carcinoma, testicular carcinoma, osteosarcoma, ovary cancer, or uterine carcinoma.

A chemotherapeutic agent according to the present invention is a substance inhibiting cell proliferation and/or inducing cell death and in a preferred embodiment further inhibits the formation of metastases.

Typically, the chemotherapeutic agent of the invention operates by inducing breaks in doubled stranded DNA.

The chemotherapeutic agent may be selected from the group of gemcitabine, telozolomid, nitrosoureas (ACNU, BCNU, CCNU, and/or HCNU)., antagonists of purine and pyrimidine bases (5-fluorouracile, 5-fluorodeoxiuridine, cytarabine and gemcitabine), cytostatic antibiotics (doxorubicin, fluorouracil, gemcitabine, procarbazine, taxol, taxotere, temozolomide, vinblastine, vincristine), camphotecine derivatives (irinotecane and topotecane), anti-estrogenes (tamoxifen, exemestane, anastrozole and fulvestrant), anti androgens (flutamide and bicalutamide) and antprogesterons (mifepriston).

Any combination of agents is also within the scope of the instant invention.

The invention further provides a method for the prognosis of an acute myelogenous leukemia or myelodisplasia secondary to a radio- or chemotherapy treatment of a neoplastic disease, comprising measuring the level of expression of the TPO receptor.

By “prognosis ” is meant the ability to predict the likely outcome of an illness, here the likelihood of onset of an acute an acute myelogenous leukemia or myelodisplasia within a period of time of less than 5 years following a treatment by radiotherapy or chemotherapy of a neoplastic disorder.

The level of expression of the TPO receptor is measured by any means well known in the art, in particular by real-time RT-PCR, flow cytometry, western blot, etc.

The level of expression is measured on platelet blood samples by flow cytometry using anti mpl antibodies or in bone marrow samples by RT-PCR in the case where patient's biopsies are taken.

The level measured is compared to a control level and if substantially lower than the control level, indicative of an increased risk of a secondary cancer namely due to genomic instability of hematopoietic stem cells.

The control level is typically the level of a population which has not been exposed to a radiotherapy used for the treatment of a neoplastic disease or a chemotherapy by an anti cancer drug of the type described above.

There is an increased risk of developing genomic instability of the I-IPCs when the level measured for the subject is at least 30%, advantageously at least 40% and most preferred at least 50%, below the level of the control population.

The instant invention also relates to a method for treating neoplastic disease other than a myeloid hemopathy by irradiation and/or administration of a chemotherapeutic agent comprising administering to a subject prior to said irradiation and/or administration of said chemotherapeutic agent a composition comprising an effective amount of TPO or active fragment thereof or an agonist of the TPO or active fragment thereof receptor.

The term “treatment or a method of treating” or its equivalent, when applied to the composition of the invention refers to a procedure or course of action that is designed to reduce or eliminate the occurrence of a myeloid hemopathy secondary to a radio- or chemotherapy treatment.

The term treatment when applied to a solid tumor or blood cancer means a reduction of the number of cancer cells in a patient, or alleviation of the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated.

Doses of the TPO or active fragment thereof or TPO-R agonist of the present invention in a pharmaceutical dosage unit or units as described above will be an efficacious, nontoxic quantity preferably selected from the range of 0.001-100 mg/kg of active compound, preferably 0.002-50 mg/kg.

The form and route of administration will depend on the nature of the active ingredient, namely of the peptidic or non peptidic nature thereof.

Preferred forms of parenteral administration include topically, rectally, transdermally, or by injection. Oral dosage units for human administration suitably contain from 0.05 to 3500 mg, suitably from 0.1 to 3000 mg, suitably from 10 to 200 mg of each active compound.

Optimal dosages to be administered may be readily determined by those skilled in the art, and will vary with the particular agent or agents in use, the strength of the preparation, the mode of administration, and the level of expression of the TPO receptor. Additional factors depending on the particular patient being treated will result in a need to adjust dosages, including patient age, weight, diet and time of administration.

The method of this invention of treating cancer comprises administering to a subject in need thereof a therapeutically effective amount of pharmaceutically active compositions of the present invention.

The invention will be further illustrated by the following figures and examples. However, these examples and figures should not be interpreted in any way as limiting the scope of the present invention.

### FIGURES

FIG. 1: Impaired DNA repair in Mpl^{-/-} and Mpl^{+/-} HSPCs.

(A) Survival Kaplan-Meier curves of 8 week-old WT and Mpl^{-/-} mice exposed to 8 and 9 Gy TBI (n=3). Means+SEM percentage of γH2AX positive of (B) WT and Mpl^{-/-} LSKs 24 h after 2 Gy TBI or not (NIR) (n=5 (NIR), n=4 (IR)) or (C) WT, Mpl^{+/-} and Mpl^{-/-} LSKs irradiated in vitro and cultured in complete medium, n=3. (D) Chromosomal aberrations in metaphases prepared from WT and Mpl^{-/-} LSKs exposed to IR (2 Gy) in vitro and cultured in complete medium or without TPO for 48 h. Left: Means+SEM of aberrations/cell from 3 independent experiments with 3 WT or Mpl^{-/-} mice. At least 50 complete metaphases scored. Right: representative images with aberrations indicated by arrows.

FIG. 2: TPO increases the DNA repair of HSPCs after TBI in vivo

gH2AX staining in LSKs (A-B) isolated from mice treated 5 h (A) or 24 h (B) before with PBS, TPO (8 μg/kg) or romiplostim (100 μg/kg) and 2 Gy TBI (A) or injected with doxorubicine (3 or 10 mg/kg) or etoposide (4 mg/kg) (B) or (C) treated 24 h before with 800 μg/kg of anti-TPO and 2 Gy TBI (n=5). Results are normalized to the means of γH2AX positive cells from PBS-injected animals.

FIG. 3: TPO confers HSPC long-term protection and genomic stability in response to IR

(A) Frequency of gH2AX-potitive LSK and LSK-CD34⁻ cells at stage 1 from mice which received PBS (IR+PBS) or TPO (IR+TPO) or not treated (NIR). Each dot represents an individual mouse. Means±SEM are shown with the values above the dots indicating mean numbers. (B) Frequencies of chromosomal aberrations in metaphases from Lin⁻ progenitors at stage 1 (n=3 mice for each group). (C-E) Analysis at stage 2. (C) Frequencies of chromosomal aberrations in metaphases from CD45.1⁺ donor-derived Lin⁻ progenitors. Means+SEM of 3 independent pools containing 3 mice per group. CD45.1 chimerism in peripheral blood (D) and in BM LSKs 4 months post-transplantation (E) Means and SEM are shown (n=7-9 mice per group).

FIG. 4: TPO regulates a DNA-PK-dependent pathway

Percentages of γH2AX positive cells in (A) LSK-CD34⁻ cells cultured in the presence or absence of 10 μm NU7441 and irradiated (2 Gy) in vitro 24 h before (means+SEM of 3 separate experiments with 4-6 mice) or (B) in LSKs from SCID mice treated with PBS or TPO (8 μg/kg) and TBI (2 Gy) 16 h before (means+SEM, n=4). (C) p-DNA-PK foci in UT7-Mpl cultured with EPO or TPO peptide (3 nM) and IR (2 Gy). Right: Representative view of pSer2056-and total DNA-PK staining (bar 10 μm), Left: Repartition of pSer2506-DNA-PK foci numbers per cell 5 min after IR. Representative experiment out of 3 performed with more than 50 cells scored. (D) Quantification of NHEJ activity in 2 Gy IR WT or Mpl^{-/-} Lin⁻ kit⁺ cells, expressed as ratio between double positive GFP⁺DsRed⁺ cells to total DsRed cells (n=8).

### EXAMPLES

Material & Methods

Mice, Cell Culture and Reconstitution Assays

Mpl^{-/-} backcrossed on C57BL/6 (CD45.2) background (kindly provided by Dr F. de Sauvage) were described previously (Petit-Cocault et al., 2007). C57BL/6J (CD45.2 and CD45.1) and CB17-Prkdc-scid mice were purchased from Charles River and Harlan laboratories, respectively. Unless otherwise specified, mice of 8-10 weeks of age were used. Lin⁻ progenitors were isolated from BM and stained as described (Saulnier et al., 2012). LSK and LSK-CD34⁻ cells were sorted using ARIAS cell sorter (BD Franklin Lakes, N.J., USA) and cultured in StemSpan SFEM (StemCell Technologies) supplemented with recombinant Flt3-Ligand (100 ng/ml), interleukin-3 (IL-3, 10 ng/ml), interleukin-6 (IL-6, 10 ng/ml), Stem cell factor (SCF, 100 ng/ml), and with or without 50 ng/ml TPO (complete medium), all from Peprotech (Rocky Hill, N.J., USA). UT7-Mpl cells (Hamelin et al., 2006) were cultured in α-minimal essential medium (α-MEM) supplemented with 10% FCS and 2 U/ml erythropoietin (EPO, Boehringer Ingelheim). Cell IR in vitro were carried-out in a Biobeam 8000 irradiator (Gamma Service Medical GmbH, Leipzig, Germany). Mice TBI were performed in cesium irradiator IBL 637 (Curie Institute, Paris). Etoposide and doxorubicine were purchased from sigma (Saint-Louis, Mo., USA), drugs were injected intraperitoneally at the indicated doses. For competitive reconstitution experiments, 3000 LSKs sorted from C57BL/6 CD45.1 mice subjected or not to TBI (2 Gy) and TPO injection (8 μg/kg body weight), were injected in lethally irradiated (10 Gy) C57BL/6 CD45.2 congenic mice, together with 3×10⁵ BM CD45.2 competitor cells and analyzed 4 months later.

Immunofluorescence

Immunofluorescence was performed as described (Pawlikowska et al., 2010) with cells cytospun on glass slides. Slides were visualized with Leica DMI 6000 (Wetzlar, Germany) microscope equipped with a 63×1.6 oil-immersion objective and a coupled device camera MicroMAX (Princeton Instruments Trenton, N.J., USA). Pictures were analyzed using ImageJ software.

Cytogenetic Assays

Chromosomal aberration analysis in metaphase spreads were performed as described previously (Pawlikowska et al., 2010). FISH-3 painting on performed using whole chromosome probes for mouse chromosomes 2 (FITC), 6 (Texas Red) and 12 (FITC:Texas Red) (MetaSystems, Altlussheim, Germany) and counterstaining with DAPI/Antifade solution (Qbiogene, USA), as reported (Pouzoulet et al., 2007). Fluorescence was analyzed with a Zeiss Axioplan epifluorescent microscope and ISIS/M-FISH imaging system (MetaSystems).

NHEJ Assays

WT and Mpl^{-/-} FACS-sorted Lin⁻Kit⁺ cells were cultured in StemSpan SFEM with cytokines (25 ng/ml SCF, 25 ng/ml IL-11, 10 ng/ml IL-3, 50 ng/ml TPO, 10 ng/ml GM-CSF). 24 h later, cells were irradiated (2 Gy) and electroporated with 1.5 μg of HindIII-digested pEGFP-Pem1-plasmid (Seluanov et al., 2004) and 0.5 μg of pDsRed-Express (Clontech Laboratories, Mountain View, Calif., USA) using Amaxa kit V. Cells were analyzed by flow cytometry 24 h later.

Statistical Analysis

Results were evaluated using either one-way ANOVA and Tukey comparison test or unpaired t-test by GraphPad Prism™ version 5.0 software (GraphPad Software Inc., San Diego, Calif., USA). For FISH analysis, a specific chi-square test adapted to Poisson statistics was used (Pouzoulet et al., 2007). Results are shown as means and SEM and the value of *P<0.05 was determined as significant, **P<0.01 or ***P<0.001 as highly significant

Results

Increased IR Sensitivity and Impaired DNA Repair in Mpl−/− HSPCs

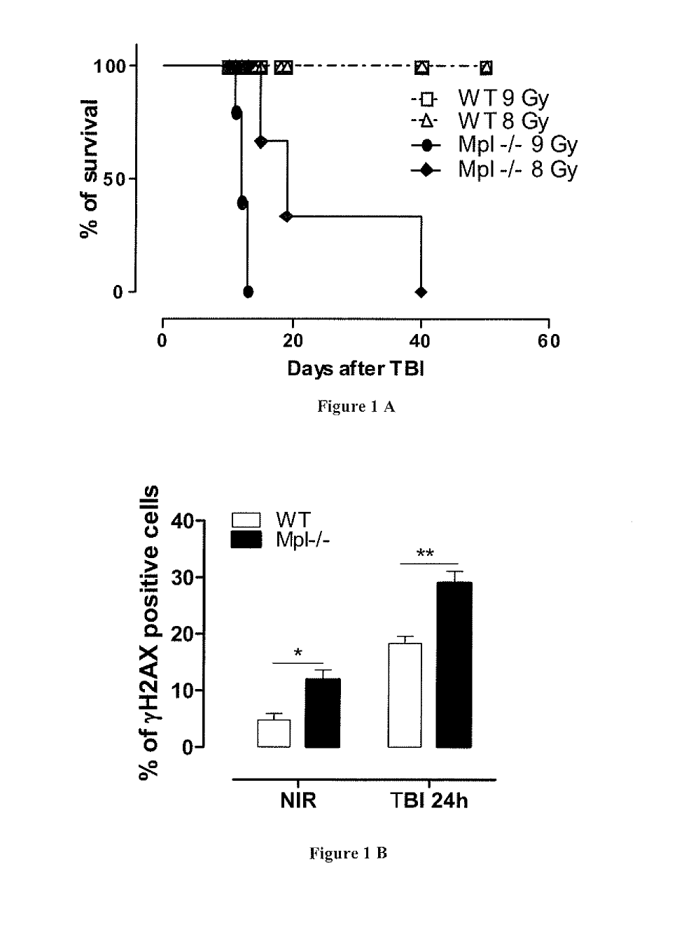

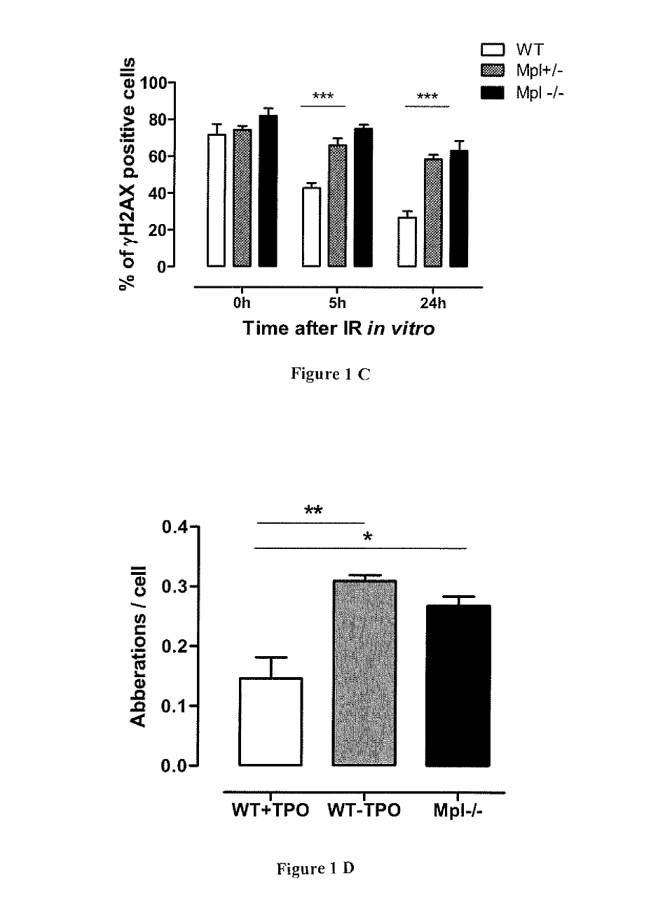

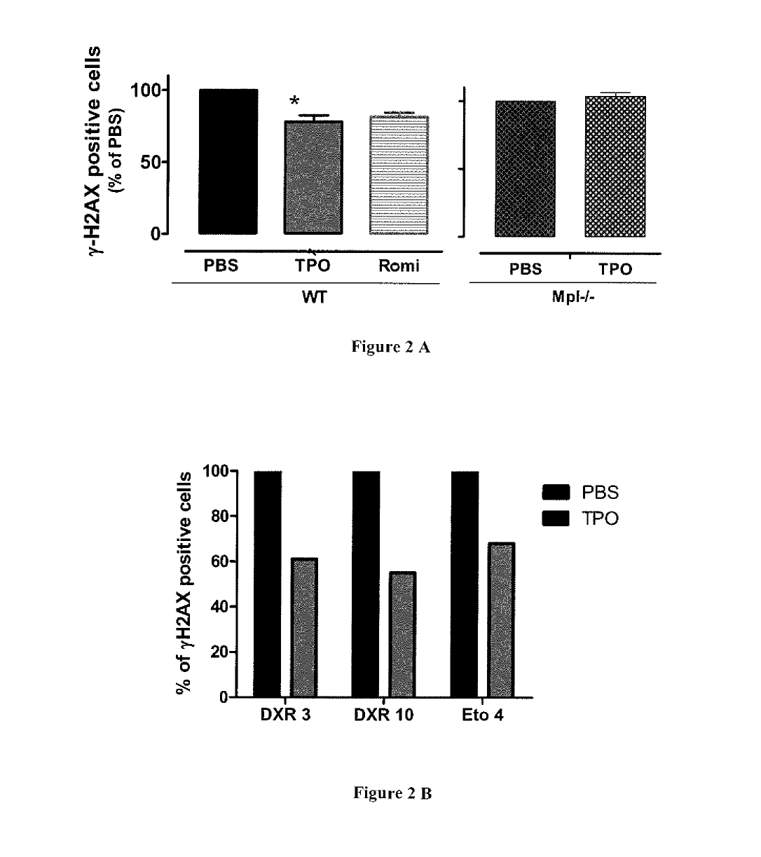

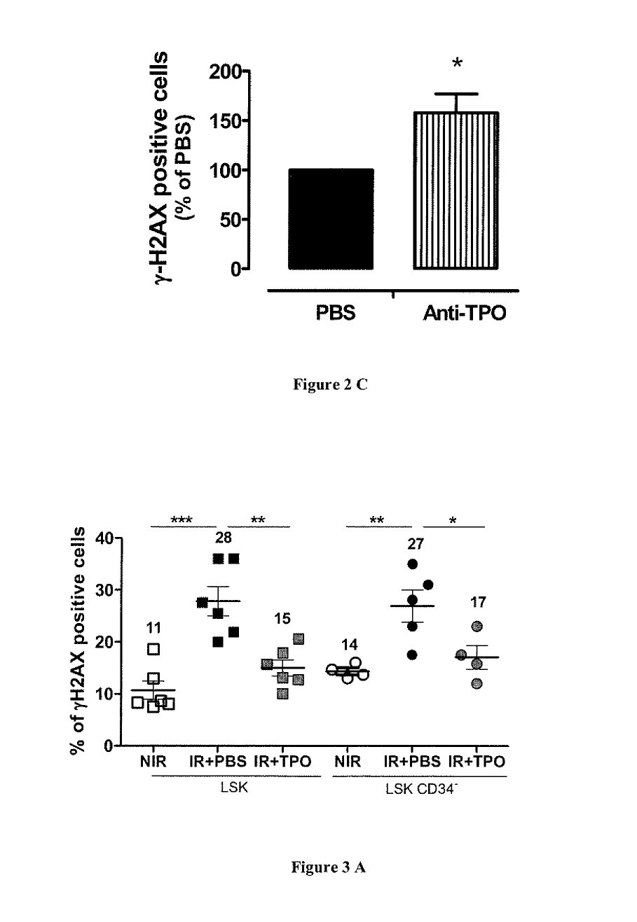

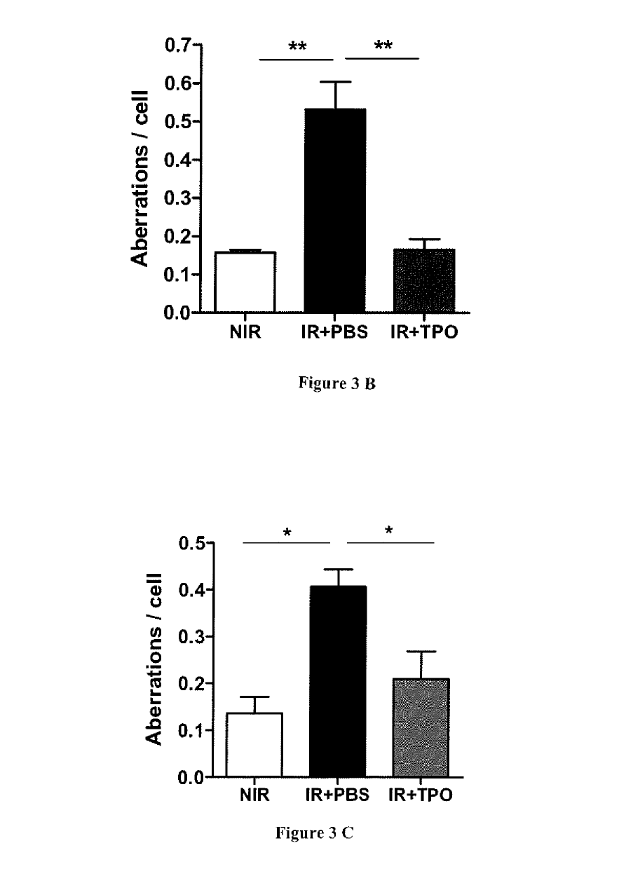

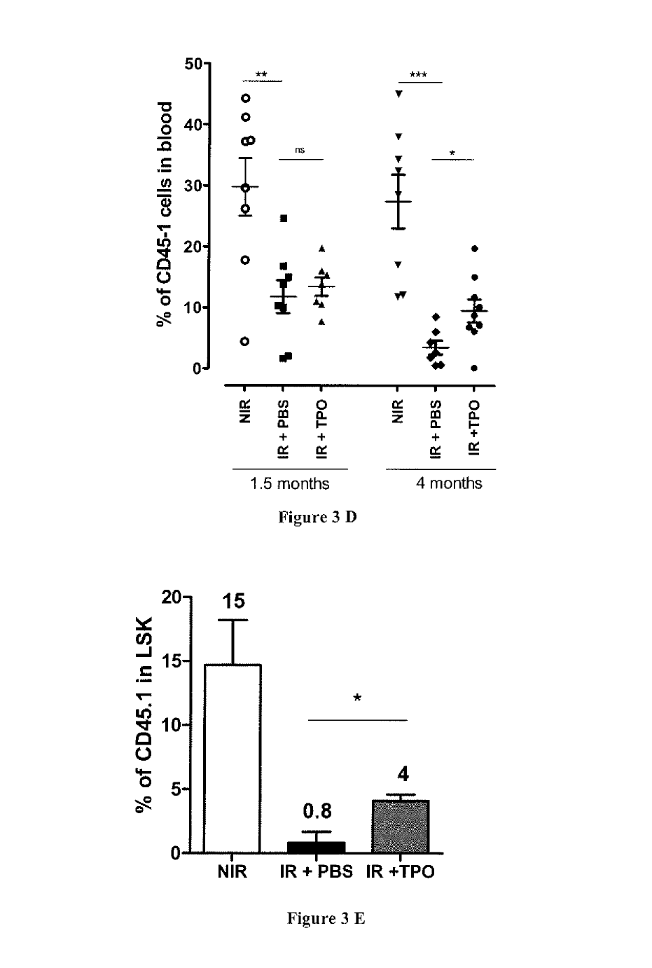

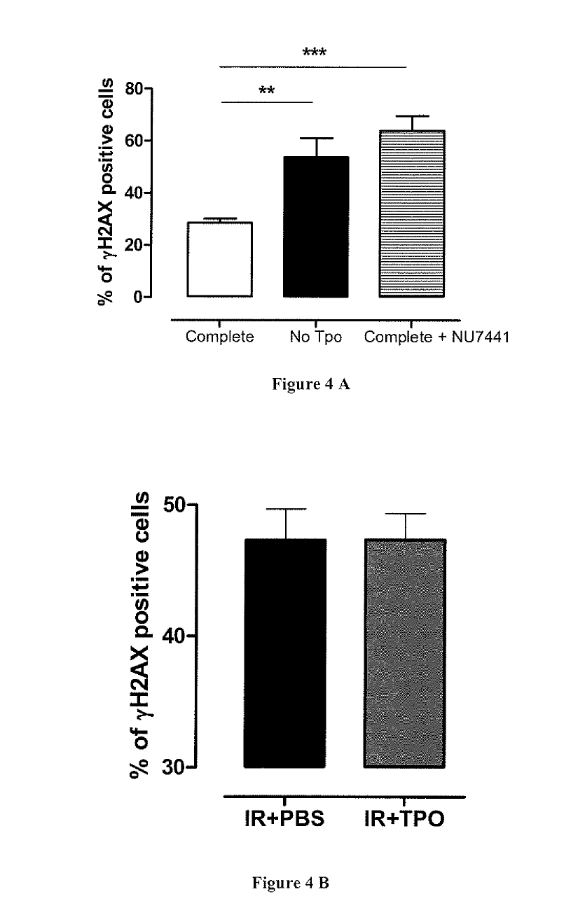

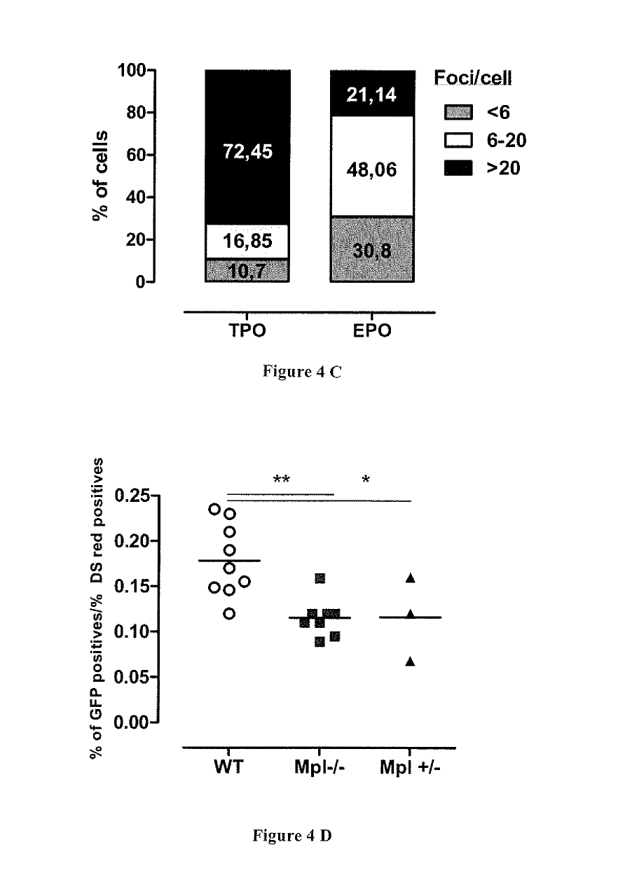

Previous studies have reported the difficulty to transplant Mpl^{-/-} mice (Wicke et al., 2010). To analyze this further, WT and Mpl^{-/-} mice were subjected to various doses of total body IR (TBI). While the doses of 9 and 8 Gy were clearly sublethal for wild-type (WT) mice they induced a rapid death of all Mpl^{-/-} mice (FIG. 1A). Clonogenic assays showed that Mpl^{-/-}Lin⁻Sca⁺Kit⁺ cells (hereafter referred to as HSPCs or LSK cells) were less resistant than WT LSKs to low doses of IR ex vivo, indicating a radiosensitive phenotype. The dose of 2 Gy was chosen to examine further this phenotype.

WT and Mpl^{-/-} LSKs showed similar low degree of caspase 3/7 activation 24 h post-IR (2 Gy) in vitro. Likewise, a low and similar percentage of WT and Mpl^{-/-} LSKs isolated 24 h following 2 Gy TBI stained positive for annexin V. Thus, the Mpl^{-/-} HSPC radiosensitivity is not due to increased IR-induced apoptosis. We could not detect senescence by SA-βgalactosidase staining in either WT or Mpl^{-/-} LSKs.

We then asked whether the lower radioresistance of Mpl^{-/-} HSPCs could be due to DNA repair defects. Thus, we first examined the presence of γH2AX nuclear foci, a commonly used surrogate marker of DSB formation. A slightly higher proportion of Mpl^{-/-} LSKs expressed spontaneous γH2AX foci as compared to WT cells, although this remained low. 24 h after TBI (2 Gy) a significantly greater number of WT LSKs harbored γH2AX foci, and this number was further increased in IR-exposed Mpl^{-/-} mice (FIG. 1B). DNA repair was assessed by analyzing γH2AX foci disappearance kinetics in vitro. As previously described (Pawlikowska et al., 2010), maximum γH2AX foci formation was observed immediately after IR and it was not significantly different for WT and Mpl^{-/-} cells. WT LSKs already started resolving their foci at 5 h post-IR and 62±3% of cells harboring foci have disappeared at 24 h. By contrast, at that time, the majority of cells still positively stained for γH2AX and about 70% of them contained 4 or more foci (FIG. 1C), indicative of prolonged DNA damage in Mpl^{-/-} HSPCs.

As Mpl^{-/-} cells, LSKs from Mpl^{+/-} mice were impaired in their capacity to resolve IR-induced γH2AX foci indicating that the level of Mpl expression is critical for correct DNA repair in response to IR.

TPO Confers Genomic Stability in Response to DNA Damage

Defects in DNA leads to a dramatic increase of large deletions and. This suggests that the loss of Mpl/TPO signal may lead to enhanced generation of misrepaired HSPCs. Indeed, cells derived from Mpl^{-/-} LSKs which were exposed to IR in vitro showed significantly higher chromosomal aberrations than WT controls (FIG. 1D). Spectral karyotyping analysis using FISH probes specific for chromosomes 2, 6 and 12 showed that BM cells isolated from Mpl^{-/-} mice 24 h post-TBI displayed 3 times more chromosomal translocations than those from WT mice No significant difference was found between WT and Mpl^{-/-} mice in the absence of IR

Lin⁻ progenitors isolated from WT mice 5 months post-TBI showed increased genomic instability as compared to their non-irradiated age-matched counterparts and this was further accentuated in cells from Mpl^{-/-} mice. This confirms the increased genomic instability in Mpl^{-/-} cells and shows that a greater number of Mpl^{-/-} cells expressing more aberrations could persist for several months in vivo.

TPO/Mpl Signaling Specifically Regulates DSB Repair in HSCs and HSPCs

To exclude the possibility that the constitutive lack of Mpl leads to increased DNA damage due to chronic changes, we analyzed the effect of acute TPO signaling upon IR. Cells were irradiated in complete medium or without TPO. The defect was similar in extent to that observed with Mpl^{-/-} cells. TPO could also act in a human cell line expressing Mpl, UT7-Mpl, which can be grown in the presence of erythropoietin (EPO) or TPO (Hamelin et al., 2006). By contrast with TPO, removal of SCF or Flt3-ligand did not impair the kinetics of IR-induced γH2AX foci or DNA break disappearance, indicating that the effect of TPO in our cell cultures is specific.

As Mpl^{-/-}, WT HSPCs irradiated in vitro and cultured in the absence of TPO harbored high numbers of chromosomal rearrangements (FIG. 1D).

We next examined whether TPO could also favor DNA repair in vivo. TPO injection in WT but not in Mpl^{-/-} mice just before 2 Gy TBI or treatment with doxorubicine (3 or 10 mg/kg) or etoposide (4 mg/kg) reduced significantly the number of LSKs displaying γH2AX foci at 5 h or 24 h as compared to mice treated with PBS alone (FIGS. 2A and 2B). Conversely, injection of anti-TPO neutralizing antibodies increased significantly γH2AX foci formation (FIG. 2C).

5 h after TBI, the proportions of LSK and LSK-CD34⁻ cells recovered from BM of PBS or TPO-treated mice were not significantly different. Importantly, TPO treatment pre-TBI did not alter the cell cycle status of either LSK or LSK-CD34⁻ cells as compared to PBS, at a time where it already reduced γH2AX foci. Moreover, expression of the cell cycle regulators p57^{kip2} and p27^{Kip1} involved in HSC quiescence and regulated in LSKs in vivo by TPO (Quian et al., 2007; Yoshihara et al., 2007), remained unchanged after TPO injection and TBI, Thus, this effect seems to result specifically from an alteration in the DNA repair process.

Altogether, these results show that TPO reduces IR-induced DNA damage in HSPCs or HSC-enriched cells in vivo and ex vivo. This indicates that the DNA repair defect of Mpl^{-/-} and Mpl^{-/-} cells results from a specific loss of TPO-mediated signaling.

To determine if TPO could improve long-term adverse effects of IR in vivo, mice were treated with TPO or PBS prior to TBI and analyzed 3 months later. It has been reported that despite seemingly recovery of phenotypically defined HSC number and cell cycle, DNA damage may persist and HSC function remains altered several months after IR (Marusyk et al., 2009; Simonnet et al., 2009). Accordingly, both LSK and LSK-CD34⁻ cells isolated from TBI mice harbored more γH2AX foci than cells from non-treated mice (FIG. 3A) while their frequencies and proliferation/quiescence status at that time were not significantly different. TPO injection almost completely abolished persistent DNA damage in HSCs and HSPCs while it has no effect on their number and cycle. In addition, the in vitro clonogenic potential of LSKs was similar in the 3 groups of mice. Thus, TPO did not reduce the numbers of γH2AX-positive LSKs by inhibiting senescence (Wang et al., 2006) or inducing proliferation. 3 months from TBI, metaphases from progenitor cells isolated from TPO-injected mice also displayed greatly reduced chromosomal aberrations (FIG. 3B). This shows that TPO injection before TBI limits IR-induced long-lasting DNA damage and genomic instability in HSC/HSPCs.

Previous studies have shown that sublethal IR induces HSC intense cycling to reconstitute hematopoiesis following acute ablation of differentiated leukocytes (Ban and Kai, 2009; Marusyk et al., 2009; Michalak et al.). Since designation of HSCs requires contribution to hematopoiesis for at least 3 months, the results above suggest that the progenitors isolated 12 weeks (FIG. 3B) after IR may represent HSC progeny and that TPO might restrain genomic instability in these cells. However, to distinguish this hypothesis from a bystander effect (Lorimore et al., 2005) and determine if the reduced DNA damage in cells from TPO-treated mice represent HSCs capable of long-term hematopoietic reconstitution, LSKs from stage 1 were used to reconstitute lethally irradiated CD45.2 recipients. 4 months after reconstitution sorted in vivo progenitor progeny of LSKs from the group that had received TBI and PBS displayed significantly increased levels of genomic rearrangements as compared to their non-irradiated counterparts or those treated with TPO before TBI (FIG. 3C). These results support previous data showing that IR induces genomic instability in HSCs that can be transmitted to their progeny (Mohrin et al., 2010). They also indicate that TPO injection before TBI significantly reduced this effect.

Increased DNA damage is linked to HSC dysfunction (Nijnik et al., 2007; Rossi et al., 2007). We therefore assessed whether TPO could also improve the reconstitution capacity of irradiated cells. Competitive transplant experiments showed that LSKs isolated from mice that had received PBS and TBI 3 months before were far less efficient than those from non-exposed mice to out-compete recipient cells, with a mean 9-fold decrease in the level of donor contribution to recipient peripheral blood leukocytes 4 months after transplantation (FIG. 3D). TPO injection partially restored this defect with a 3- to 5-fold increase in donor CD45.1 chimerism in the peripheral blood and in BM LSK cells (FIGS. 3D and 3E), although this level remained decreased as compared to mice reconstituted with LSKs from non-irradiated donors. No difference was found between the TPO and PBS groups at 6 weeks posttransplantation (FIG. 3D) suggesting that TPO treatment improved HSC function of irradiated cells rather than their short-term proliferation.

Altogether, these results show that injection of TPO in vivo just before IR reduced IR-induced HSPCs mutagenesis and loss function.

TPO Regulates a DNA-PK-Dependent NHEJ Pathway

We next examined the mechanism involved in TPO-mediated DSB repair. HR and NHEJ are the two major DSB repair pathways. The above data show that TPO regulates DNA damage in both HSC-enriched, mostly quiescent LSK-CD34⁻ cells (65-70% G0), HSPCs (LSKs, 20-30% G0), proliferating Lin− progenitors and UT7-Mpl cell lign. This suggests that the mechanism involved is different from HR which acts only during G2/S phases. Supporting this hypothesis TPO did not increase IR-induced Rad51 foci formation, an in vivo functional marker of HR, and it could not improve repair of DSBs induced by replicative stresses such as camptotecin and hydroxyurea.

NHEJ is the predominant repair mechanism for DSBs resulting from IR (Iliakis et al., 2004). Thus, we then tested this pathway by analyzing the involvement of its main enzyme, DNA-PK. Addition of the specific DNA-PK inhibitor NU7441 to TPO-containing cultures of LSK-CD34⁻ cells completely abolished TPO-improved γH2AX foci resolution to the level observed in the absence of TPO (FIG. 4A). Moreover, by contrast with its effect in WT mice, TPO injection to SCID mice which are NHEJ repair deficient as a result of DNA-PKcs mutation did not decrease TBI-induced LSK γH2AX foci formation (FIG. 4B). This confirms the importance of DNA-PK activation in TPO-mediated DNA repair.

IR has been shown to induce phosphorylation of DNA-PK at Ser2056 and T2609 and pDNA-PK foci formation can be used as an in vivo functional marker of NHEJ activity (Chan et al., 2002; Chen et al., 2005). To test whether TPO could affect DNA-PK activation, we used the UT7-Mpl cell model since the available anti-pDNA-PK antibodies could not detect DNA-PK in mouse cells. According to previous studies (Chen et al., 2005), pSer2056-DNA-PK is rapidly induced upon IR and accumulates in nuclear foci. In the absence of TPO, the number of UT7-Mpl cells showing IR-induced pSer2056- and pT2609-DNA-PK foci was significantly decreased and cells that stained positively displayed a greatly reduced foci number, as compared to TPO-treated cells (FIG. 4C). By contrast, total DNA-PK gave a diffuse signal that did not change upon IR or TPO treatment.

To confirm activation of NHEJ repair by TPO, we examined NHEJ activity. First, we transfected WT and Mpl^{-/-} sorted-Lin⁻kit⁺ cells with a plasmid-based episomal DSB end-rejoining assay, in which the religation leads to GFP expression (Seluanov et al., 2004). This assay showed that NHEJ activity in response to IR was significantly decreased in and Mpl^{+/-} progenitors (FIG. 4D). These results show that TPO stimulates DNA-PK activity and NHEJ-mediated DNA-repair.

Throughout this application, various references describe the state of the art to which this invention pertains. The disclosures of these references are hereby incorporated by reference into the present disclosure.

1. A method of preventing or reducing genomic instability of hematopoietic stem cells incurred through radio- or chemotherapy treatment in a patient in need thereof, comprising the steps of
administering to said patient, prior to the radio- or chemotherapy treatment, a therapeutically effective amount of a pharmaceutical composition comprising
thrombopoietin (TPO) or an active fragment of thereof; or
an agonist of the TPO receptor (TPO-R),
said patient having reduced or no genomic instability in said hematopoietic stem cells after said radio or chemotherapy treatment.

2. The method of claim 1 for wherein said pharmaceutical composition is administered in a single dose.

3. The method of claim 2, wherein said single dose is administered from 60 minutes to 30 minutes before the radio-or chemotherapy treatment.

4. The method of claim 1, wherein said administering step is performed by administering said agonist, and wherein said agonist is romiplostin.

5. The method of claim 1, wherein said administering step is performed by administering said agonist, and wherein said agonist is eltrombopag.

6-13. (canceled)

14. A method for treating, by irradiation and/or administration of a chemotherapeutic agent, a neoplastic disease other than a myeloid hemopathy in a subject in need thereof, comprising
prior to said irradiation and/or administration of a chemotherapeutic agent, administering to said subject, an effective amount of thrombopoietin (TPO) or a fragment thereof or an agonist of the TPO receptor to reduce or eliminate genomic instability in hemotopoietic stem cells in said subject after said radio or chemotherapy treatment.
